(19) United States
(12) Patent Application Publication
(10) Pub. No.: US 2020/0289822 A1
(43) Pub. Date: Sep. 17, 2020

(54) THERAPEUTIC MODULATION TO TREAT ACUTE DECOMPENSATED HEART FAILURE, AND ASSOCIATED SYSTEMS AND METHODS

(71) Applicant: Nevro Corp., Redwood City, CA (US)

(72) Inventor: Tamara C. Baynham, Redwood City, CA (US)

(21) Appl. No.: 16/354,063

(22) Filed: Mar. 14, 2019

(51) Int. Cl. A61N 1/36; A61N 1/05; A61B 5/053; A61B 5/024

(52) U.S. Cl. CPC A61N 1/36114; A61N 1/36062; A61N 1/36171; A61N 1/36071; A61B 5/02405; A61N 1/36175; A61N 1/36164; A61N 1/36139; A61B 5/0531; A61N 1/0553

(57) ABSTRACT

Systems and methods for treating a patient having acute decompensated heart failure (ADHF) using electrical stimulation are disclosed. A representative method for treating a patient includes positioning an implantable signal delivery device proximate to a target location at or near the patient's spinal cord within a vertebral range of about T1 to about T12, directing an electrical therapy signal to the target location via the implantable signal delivery device, wherein the electrical signal has a frequency in a frequency range of from 1.2 kHz to 100 kHz to modulate one or more of the patient's sympathetic nerves and treat the patient's ADHF.

### TECHNICAL FIELD

The present technology is directed generally to methods and systems for treating acute decompensated heart failure (ADHF) in a patient in need thereof by applying electrical stimulation to a target neural population located within the patient's spinal cord.

### BACKGROUND

Heart failure is a growing problem worldwide with more than 20 million people around the world affected, including 5 million in the United States alone. Heart failure affects 6-10% of people over the age of 65. In the United States, the treatment of heart failure has a direct cost of over $35 billion per year, most of which results from hospitalization. There are over 1 million hospitalizations with a primary diagnosis of heart failure each year in the United States, and heart failure is the most common diagnosis for hospitalizations for patients over 65 years of age. ADHF is a sudden or gradual worsening of signs and symptoms associated with heart failure. Most often, ADHF presents as severe pulmonary and systemic congestion. Hospitalization for ADHF is a powerful predictor of readmission and post-discharge death in patients with chronic heart failure, with mortality rates as high as 20% after discharge. Accordingly, there is a need for systems and methods for reducing and/or eliminating the effects of ADHF.

### BRIEF DESCRIPTION OF THE DRAWINGS

FIG. 1A is a partially schematic illustration of an implantable spinal cord modulation system positioned at a patient's spine to deliver therapeutic signals in accordance with some embodiments of the present technology.

FIG. 1B is a partially schematic, cross-sectional illustration of a patient's spine, illustrating representative locations for implanted lead bodies in accordance with some embodiments of the present technology.

FIG. 2 is a partially schematic illustration of a patient's sympathetic and parasympathetic nervous systems, and some of the organs innervated thereby, and also illustrates a representative location for an implanted lead body in accordance with some embodiments of the present technology.

FIG. 3 is a flow diagram illustrating a method for treating ADHF in accordance with embodiments of the present technology.

### DETAILED DESCRIPTION

The present technology is directed generally to systems and methods for treating and/or reducing one or more symptoms associated with acute decompensated heart failure (ADHF), and in particular, to systems and methods for treating and/or reducing the symptoms of ADHF by modulating splanchnic nerve activity to prevent or decrease congestion and/or fluid retention. In some embodiments, the present technology includes methods for treating a patient's ADHF by positioning an implantable signal delivery device proximate to a target location at or near the patient's spinal cord within a vertebral range of about T1 to about T12, directing an electrical therapy signal having a frequency of 1.2 kHz to 100 kHz to the target location via the implantable signal delivery device, and modulating one or more of the patient's sympathetic nerves.

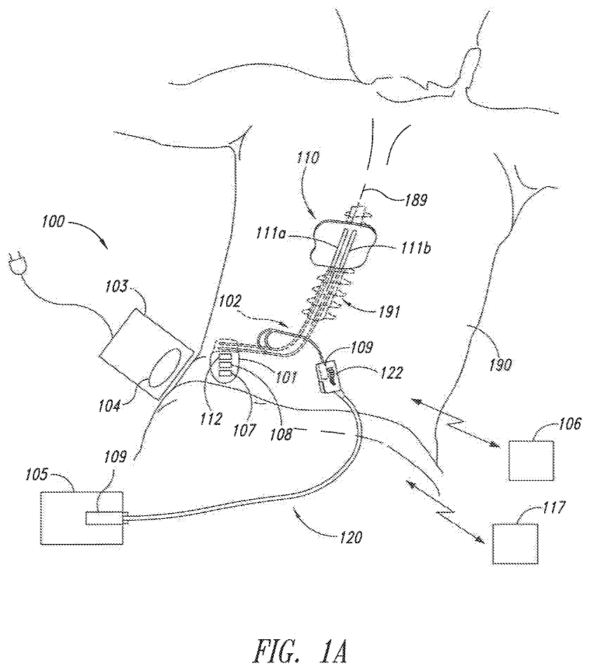

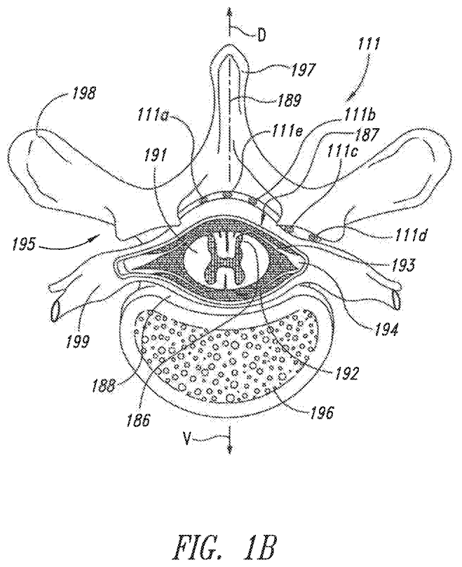

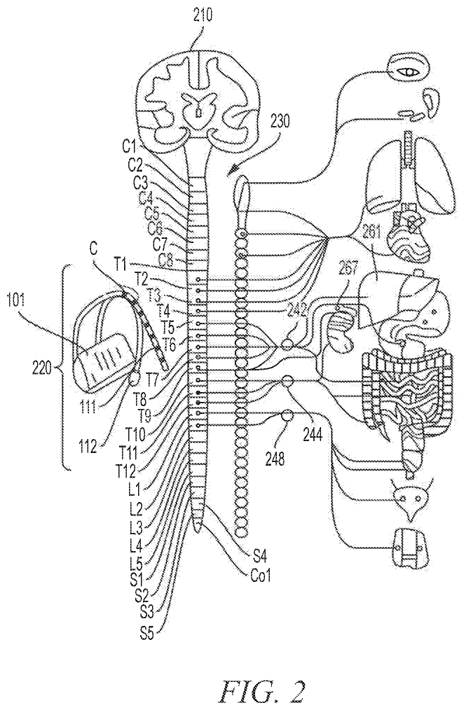

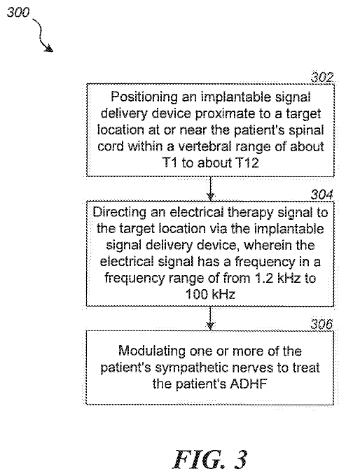

Definitions of selected terms are provided under heading 1.0 (“Definitions”). General aspects of the anatomical and physiological environment in which the disclosed technology operates are described below under heading 2.0 (“Introduction”), Representative treatment systems and their characteristics are described under heading 3.0 (“System Characteristics”) with reference to FIGS. 1A and 1B. Representative methods for treating ADHF and target locations for positioning leads are described under heading 4.0 (“Representative Methods for Treating Acute Decompensated Heart Failure”) with reference to FIGS. 2 and 3. Representative examples are described under heading 5.0 (“Representative Examples”).

### 1.0 DEFINITIONS

As used herein, the terms “therapeutic signal,” “electrical therapy signal,” “therapeutic electrical stimulation,” “therapeutic modulation,” “therapeutic modulation signal,” and “TS” refer to an electrical signal having (1) a frequency of from about 1.2 kHz to about 100 kHz, or from about 1.5 kHz to about 100 kHz, or from about 2 kHz to about 50 kHz, or from about 3 kHz to about 20 kHz, or from about 3 kHz to about 15 kHz, or from about 5 kHz to about 15 kHz, or from about 3 kHz to about 10 kHz. or 1 kHz, 2 kHz, 3 kHz, 4 kHz, 5 kHz, 10 kHz, 15 kHz, 20 kHz, 25 kHz, 50 kHz, or 100 kHz; (2) an amplitude within an amplitude range of about 0.1 mA to about 20 mA, about 0.5 mA to about 10 mA, about 0.5 mA to about 7 mA, about 0.5 mA to about 5 mA, about 0.5 mA to about 4 mA, about 0.5 mA to about 2.5 mA; (3) a pulse width in a pulse width range of from about 1 microsecond to about 333 microseconds, from about 10 microseconds to about 333 microseconds, from about 10 microseconds to about 166 microseconds, from about 25 microseconds to about 166 microseconds, from about 25 microseconds to about 100 microseconds, from about 30 microseconds to about 100 microseconds, from about 33 microseconds to about 100 microseconds, from about 50 microseconds to about 166 microseconds; from about 1 microsecond to about 50 microseconds, from about 1 microsecond to about 10 microseconds, from about 2 microseconds to about 5 microseconds, from about 5 microseconds to about 10 microseconds; (4) an interphase delay including a 30 microsecond cathodic pulse followed by a 20 microsecond interphase delay followed by a 30 microsecond anodic pulse followed by another 20 microsecond interphase delay unless otherwise stated that is delivered for therapeutic purposes; and (5) a duty cycle of 25% to 100%. Unless otherwise stated, the present disclosure includes any applicable combination of the foregoing ranges, e.g., excluding combinations of pulse width and frequency that are mathematically incompatible,

Unless otherwise stated, the terms “about” and “approximately” refer to values within 10% of a stated value.

As used herein, the terms therapy signal and therapeutic signal can refer to a first therapeutic signal, and in some embodiments, the disclosed systems can also deliver a second therapeutic signal.

As used herein, “acute decompensated heart failure” and “ADHF” refer to a sudden worsening or gradual onset of one or more signs and/or symptoms associated with heart failure, such as difficulty breathing, swelling in a patient's legs and/or feet, and fatigue. (See Joseph, S. M., et, al., “Acute Decompensated Heart Failure”, Texas Heart Institute Journal, 36(6):510-20. (2009)). These events often lead to patients seeking unplanned medical attention. (Id.) ADHF is caused by congestion of one or more organs due to fluid accumulation in the patient's body which is inadequately circulated by their heart. (Id.) Pulmonary and systemic congestion is a common finding in patients having ADHF and is often caused by increased left- and right-heart filling pressures. (Id.) These events can lead to acute respiratory distress. (Id.) In addition, ADHF can be caused by myocardial infarction, abnormal heart rhythm, infection, and/or thyroid disease. (Id.) The systems and methods of the present technology are configured to treat ADHF by modulating the patient's splanchnic nerve activity to (1) prevent and/or decrease sympathetic congestion (e.g., reduce mobilization of venous reserves), splanchnic congestion (e.g., by reducing the patient's retention of sodium and/or fluid), fluid retention, lung congestion, circulatory volume, and/or edema, (2) increase the patient's splanchnic circulation, and/or (3) otherwise increase activity of the patient's nervous system.

“Treating” or “treatment” as used herein with regard to ADHF refers to preventing progression and/or onset of ADHF, ameliorating, reducing, eliminating, suppressing, and/or alleviating ADHF, and/or one or more of the symptoms associated with ADHF, generating a complete or partial regression of ADHF, or any suitable combination thereof. “Treating” or “treatment” also refers to reducing a patient's pain.

As used herein, and unless otherwise noted, the terms “modulate,” “modulation,” “stimulate,” and “stimulation” refer generally to signals that have any of the foregoing effects. Accordingly, a spinal cord “stimulator” can have an inhibitory effect on certain neural populations.

The following terms are used interchangeably throughout the present disclosure: electrical signal, therapeutic modulation signal, therapeutic signal, electrical pulse, signal, waveform, modulation signal, modulation, neural modulation signal, and therapeutic electrical signal.

### 2.0 INTRODUCTION

The present technology is directed generally to spinal cord modulation and associated systems and methods for treating a patient's ADHF and/or symptoms associated with ADHF with a therapeutic signal delivered from one or more therapeutic electrical signal elements or components. The systems and methods described herein may treat ADHF generally without generating paresthesia, which may or may not be a side effect. Additional side effects can include unwanted motor stimulation or blocking, and/or interference with sensory functions other than ADHF and/or associated symptoms. Some embodiments continue to provide ADHF for at least some period of time after the modulation signals have ceased. Although some embodiments are described below with reference to modulating the dorsal column, dorsal horn, dorsal root, dorsal root entry zone, and other particular regions of the spinal column to treat ADHF, the modulation may, in some instances be generally directed to the patient's thoracic region (e.g., T1-T12, such as T5-T12) of the spinal column which may further include combination placement (e.g., spanning more than one thoracic region such as T2-T4 and/or T5-T7) rather than individually at T1, T2, T3, or T4, and so on. In addition, the modulation may, in some instances, be directed to the splanchnic nerve itself and/or nerves associated with the splanchnic nerve. In any of these embodiments, the technology can include chronic stimulation and/or intermittent stimulation.

Specific details of some embodiments of the present technology are described below with reference to methods for modulating one or more target neural populations within the patient's spinal cord, and associated implantable structures for providing the modulation. Some embodiments can have configurations, components and/or procedures different than those which are described herein, and other embodiments may eliminate particular components or procedures. A person of ordinary skill in the relevant art, therefore, will understand that the present disclosure may include some embodiments with additional elements, and/or may include some embodiments without several of the features shown and described below with reference to FIGS. 1A-3.

Also provided herein are various embodiments of neuromodulation systems, methods, and therapies for treating ADHF. Unless otherwise specified, the specific embodiments discussed are not to be construed as limitations on the scope of the disclosed technology. It will be apparent to one skilled in the relevant art that various equivalents, changes, and modifications may be made without departing from the scope of the disclosed technology, and it is understood that such equivalent embodiments are to be included herein.

In general terms, the present technology is directed to producing a therapeutic effect that includes reducing or eliminating ADHF and/or one or more symptoms thereof in the patient. The therapeutic effect can be produced by inhibiting, suppressing, downregulating, preventing, and/or otherwise modulating the activity of the affected and/or target neural population, such as a target neural population in the thoracic region of the patient's spinal column (e.g., T1-T12) which may further include combination placement (e.g., spanning more than one thoracic region such as T2-T4 and/or T5-T7) rather than individually at T1, T2, T3, or T4, and so on. In addition, the modulation may, in some instances, be directed to the splanchnic nerve itself and/or nerves associated with the splanchnic nerve. In any of these embodiments, the technology can include chronic stimulation and/or intermittent stimulation. In some embodiments, the affected neural population is located within, proximate to, or otherwise corresponds to the patient's sympathetic nervous system, which modulates the patient's hemodynamic system. Without intending to be bound by any particular theory, inhibiting at least a portion of the patient's sympathetic nervous system, such as one or more sympathetic nerves corresponding to (1) the patient's heart, (2) the patient's lungs, or (3) both (1) and (2), may result in the therapeutic effect by modulating the patient's splanchnic nerve activity so as to prevent and/or decrease one or more of the following: sympathetic congestion, fluid retention, lung congestion, and edema, and/or increase the patient's splanchnic circulation, and/or otherwise alter activity of the patient's nervous system. In some embodiments, the therapeutic effect can be produced by inhibiting one or more sympathetic nerves associated with one or more thoracic vertebrae, such as T1 to T12, and/or the renal nerve. For example, one or more sympathetic nerves are sympathetic nerves that are associated with the patient's circulation, such as the greater splanchnic nerve, the lesser splanchnic nerve, the least splanchnic nerve, and/or the renal nerve. In some embodiments, therapeutic effect can be produced by modulating one or more sympathetic nerves which reduces mobilization of venous reservoirs, reduces splanchnic congestion, and/or reduces the patient's effective circulatory volume. For example, the patient's splanchnic congestion can be reduced by reducing the patient's retention of sodium and/or fluid.

It is expected that the techniques described below with reference to FIGS. 1A-3 can produce more effective, more robust, less complicated and/or otherwise more desirable results than can existing stimulation therapies and/or other ADHF therapies. In particular, these techniques can produce results that reduce or eliminate ADHF and/or one or more symptoms associated with ADHF. These results may persist after the modulation signal ceases. In addition, techniques corresponding to the present technology can be performed by delivering modulation signals continuously or intermittently (e.g., on a schedule) to obtain a beneficial effect with respect to treating ADHF and/or one or more symptoms associated with ADHF.

Many of the following embodiments are directed to producing a therapeutic effect that includes treating ADHF and/or one or more symptoms associated with ADHF in a patient. The therapeutic effect can be produced by inhibiting, suppressing, downregulating, preventing, and/or otherwise modulating the activity of the affected neural population.

In some embodiments, therapeutic modulation signals are directed to the target location that generally includes at least a portion of the patient's spinal cord, e.g., the dorsal column of the patient's spinal cord. The modulation signals can be directed to the dorsal horn, dorsal root, dorsal root ganglion, dorsal root entry zone, and/or other particular areas at or in close proximity to the spinal cord itself. The foregoing areas are referred to herein collectively as the spinal cord region. In some embodiments, therapeutic modulation signals are directed generally to the thoracic region of the patient's spinal cord, for example, at T5 to T12, which may further include combination placement (e.g., spanning more than one thoracic region such as T2-T4 and/or T5-T7) rather than individually at T1, T2, T3, or T4, and so on. In addition, the modulation may, in some instances, be directed to the splanchnic nerve itself and/or nerves associated with the splanchnic nerve. In any of these embodiments, the technology can include chronic stimulation and/or intermittent stimulation.

Without being bound by the following theories, or any other theories, it is expected that the therapy signals act to address ADHF and/or one or more symptoms associated with ADHF via one or both of two mechanisms: (1) by modulating neural transmissions entering the sympathetic nervous system, and/or (2) by modulating neural activity at the sympathetic nerves themselves. The presently disclosed therapy is expected to treat ADHF and/or one or more symptoms associated with ADHF without the side effects generally associated with conventional SCS therapies, e.g., including but not limited to, SCS therapies conducted below 1200 Hz and including paresthesia, or other therapies conventionally used to treat ADHF and/or one or more symptoms associated with ADHF. Several SCS therapies are discussed further in U.S. Pat. No. 8,170,675, incorporated herein by reference. These and other advantages associated with embodiments of the presently disclosed technology are described further below.

### 3.0 SYSTEM CHARACTERISTICS

FIG. 1A schematically illustrates a representative patient therapy system 100 for treating a patient having ADHF, arranged relative to the general anatomy of the patient's spinal column 191. The system 100 can include a signal generator 101 (e,g., an implanted or implantable pulse generator or IPG), which may be implanted subcutaneously within a patient 190 and coupled to one or more signal delivery elements or devices 110. The signal delivery elements or devices 110 may be implanted within the patient 190, at or off the patient's spinal cord midline 189, The signal delivery elements 110 carry features for delivering therapy to the patient 190 after implantation. The signal generator 101 can be connected directly to the signal delivery devices 110, or it can be coupled to the signal delivery devices 110 via a signal link, e.g., a lead extension 102. In some embodiments, the signal delivery devices 110 can include one or more elongated lead(s) or lead body or bodies 111 (identified individually as a first lead 111a and a second lead 111b). As used herein, the terms “signal delivery device,” “lead,” and/or “lead body” include any of a number of suitable substrates and/or supporting members that include or carry electrodes/devices for providing therapy signals to the patient 190. For example, the lead or leads 111 can include one or more electrodes or electrical contacts that direct electrical signals into the patient's tissue, e.g., to provide for therapeutic relief. In some embodiments, the signal delivery elements 110 can include structures other than a lead body (e.g., a paddle) that also direct electrical signals and/or other types of signals to the patient 190, e.g., as disclosed in U.S. patent application Ser. No. 15/915,339, which is incorporated herein by reference in its entirety.

In some embodiments, one signal delivery device may be implanted at a first location within T5-T12, and a second signal delivery device may be implanted at a second location within T1-T12. The first lead 111a and/or the second lead 111b shown in FIG. 1A may be positioned just off the spinal cord midline 189 (e.g., about 1 mm offset). In some embodiments, the leads 111 may be implanted at a vertebral level ranging from, for example, about T1 to about T12. In some embodiments, one or more signal delivery devices can be implanted at other vertebral levels, e.g., as disclosed in U.S. Pat. No. 9,327,121, which is incorporated herein by reference in its entirety.

The signal generator 101 can transmit signals (e.g., electrical signals) to the signal delivery elements 110 that excite and/or suppress target nerves (e.g., sympathetic nerves). The signal generator 101 can include a machine-readable (e.g., computer-readable) or controller-readable medium containing instructions for generating and transmitting suitable therapy signals. The signal generator 101 and/or other elements of the system 100 can include one or more processor(s) 107, memory unit(s) 108, and/or input/output device(s) 112. Accordingly, the process of providing modulation signals, providing guidance information for positioning the signal delivery devices 110, establishing battery charging and/or discharging parameters, and/or executing other associated functions can be performed by computer-executable instructions contained by, on or in computer-readable media located at the pulse generator 101 and/or other system components. Further, the pulse generator 101 and/or other system components may include dedicated hardware, firmware, and/or software for executing computer-executable instructions that, when executed, perform any one or more methods, processes, and/or sub-processes described in the materials incorporated herein by reference. The dedicated hardware, firmware, and/or software also serve as “means for” performing the methods, processes, and/or sub-processes described herein. The signal generator 101 can also include multiple portions, elements, and/or subsystems (e.g., for directing signals in accordance with multiple signal delivery parameters), carried in a single housing, as shown in FIG. 1A, or in multiple housings.

The signal generator 101 can also receive and respond to an input signal received from one or more sources. The input signals can direct or influence the manner in which the therapy, charging, and/or process instructions are selected, executed, updated, and/or otherwise performed. The input signals can be received from one or more sensors (e.g., an input device 112 shown schematically in FIG. 1A for purposes of illustration) that are carried by the signal generator 101 and/or distributed outside the signal generator 101 (e.g., at other patient locations) while still communicating with the signal generator 101. The sensors and/or other input devices 112 can provide inputs that depend on or reflect patient state (e.g., patient position, patient posture, and/or patient activity level), and/or inputs that are patient-independent (e.g., time). Still further details are included in U.S. Pat. No. 8,355,797, incorporated herein by reference in its entirety.

In some embodiments, the signal generator 101 and/or signal delivery devices 110 can obtain power to generate the therapy signals from an external power source 103. For example, the external power source 103 can bypass an implanted signal generator and generate a therapy signal directly at the signal delivery devices 110 (or via signal relay components). The external power source 103 can transmit power to the implanted signal generator 101 and/or directly to the signal delivery devices 110 using electromagnetic induction (e.g., RF signals). For example, the external power source 103 can include an external coil 104 that communicates with a corresponding internal coil (not shown) within the implantable signal generator 101, signal delivery devices 110, and/or a power relay component (not shown). The external power source 103 can be portable for ease of use.

In some embodiments, the signal generator 101 can obtain the power to generate therapy signals from an internal power source, in addition to or in lieu of the external power source 103. For example, the implanted signal generator 101 can include a non-rechargeable battery or a rechargeable battery to provide such power. When the internal power source includes a rechargeable battery, the external power source 103 can be used to recharge the battery. The external power source 103 can in turn be recharged from a suitable power source (e.g., conventional wall power).

During at least some procedures, an external stimulator or trial modulator 105 can be coupled to the signal delivery elements 110 during an initial procedure, prior to implanting the signal generator 101. For example, a practitioner (e.g., a physician and/or a company representative) can use the trial modulator 105 to vary the modulation parameters provided to the signal delivery elements 110 in real time, and select optimal or particularly efficacious parameters. These parameters can include the location from which the electrical signals are emitted, as well as the characteristics of the electrical signals provided to the signal delivery devices 110. In some embodiments, input is collected via the external stimulator or trial modulator 105 and can be used by the practitioner to help determine which parameters to vary. In a typical process, the practitioner uses a cable assembly 120 to temporarily connect the trial modulator 105 to the signal delivery device 110. The practitioner can test the efficacy of the signal delivery devices 110 in an initial position. The practitioner can then disconnect the cable assembly 120 (e.g., at a connector 122), reposition the signal delivery devices 110, and reapply the electrical signals. This process can be performed iteratively until the practitioner obtains the desired position for the signal delivery devices 110. Optionally, the practitioner may move the partially implanted signal delivery devices 110 without disconnecting the cable assembly 120. Furthermore, in some embodiments, the iterative process of repositioning the signal delivery devices 110 and/or varying the therapy parameters may not be performed.

The signal generator 101, the lead extension 102, the trial modulator 105 and/or the connector 122 can each include a receiving element 109. Accordingly, the receiving elements 109 can be patient-implantable elements, or the receiving elements 109 can be integral with an external patient treatment element, device or component (e.g., the trial modulator 105 and/or the connector 122). The receiving elements 109 can be configured to facilitate a simple coupling and decoupling procedure between the signal delivery devices 110, the lead extension 102, the pulse generator 101, the trial modulator 105 and/or the connector 122. The receiving elements 109 can be at least generally similar in structure and function to those described in U.S. Patent Application Publication No. 2011/0071593, which is incorporated by reference herein in its entirety.

After the signal delivery elements 110 are implanted, the patient 190 can receive therapy via signals generated by the trial modulator 105, generally for a limited period of time. During this time, the patient wears the cable assembly 120 and the trial modulator 105 outside the body. Assuming the trial therapy is effective or shows the promise of being effective, the practitioner then replaces the trial modulator 105 with the implanted signal generator 101, and programs the signal generator 101 with therapy programs selected based on the experience gained during the trial period. Optionally, the practitioner can also replace the signal delivery elements 110. Once the implantable signal generator 101 has been positioned within the patient 190, the therapy programs provided by the signal generator 101 can be updated remotely via a wireless physician's programmer 117 (e.g., a physician's laptop, a physician's remote or remote device, etc.) and/or a wireless patient programmer 106 (e.g., a patient's laptop, patient's remote or remote device, etc.). Generally, the patient 190 has control over fewer parameters than the practitioner. For example, the capability of the patient programmer 106 may be limited to starting and/or stopping the signal generator 101 and/or adjusting the signal amplitude. The patient programmer 106 may be configured to accept pain relief input as well as other variables, such as medication use.

In some embodiments, the present technology includes receiving patient feedback, via a sensor, that is indicative of, or otherwise corresponds to, the patient's response to the signal. Feedback includes, but is not limited to, motor, sensory, and verbal feedback. In response to the patient feedback, one or more signal parameters can be adjusted, such as frequency, pulse width, amplitude or delivery location.

FIG. 1B is a cross-sectional illustration of the spinal cord 191 and an adjacent vertebra 195 (based generally on information from Crossman and Neary, “Neuroanatomy,” 1995 (published by Churchill Livingstone)), along with multiple leads 111 (shown as leads 111a-111e) implanted at representative locations. For purposes of illustration, multiple leads 111 are shown in FIG. 1B implanted in a single patient. In addition, for purposes of illustration, the leads 111 are shown as elongated leads however, leads 111 can be paddle leads. In actual use, any given patient will likely receive fewer than all the leads 111 shown in FIG. 1B.

The spinal cord 191 is situated within a vertebral foramen 188, between a ventrally located ventral body 196 and a dorsally located transverse process 198 and spinous process 197. Arrows V and D identify the ventral and dorsal directions, respectively. The spinal cord 191 itself is located within the dura mater 199, which also surrounds portions of the nerves exiting the spinal cord 191, including the ventral roots 192, dorsal roots 193 and dorsal root ganglia 194. The dorsal roots 193 enter the spinal cord 191 at the dorsal root entry portion 187, and communicate with dorsal horn neurons located at the dorsal horn 186. In some embodiments, the first and second leads 111a, 111b are positioned just off the spinal cord midline 189 (e.g., about 1 mm offset) in opposing lateral directions so that the two leads 111a, 111b are spaced apart from each other by about 2 mm, as discussed above, with the first lead illa positioned at a location within T1 -T4 and the second lead 111b positioned at a location within T5-T12. In some embodiments, a lead or pairs of leads can be positioned at other locations, e.g., toward the outer edge of the dorsal root entry portion 187 as shown by a third lead 111c, or at the dorsal root ganglia 194, as shown by a fourth lead 111d, or approximately at the spinal cord midline 189, as shown by a fifth lead 111e.

In some embodiments, the devices and systems of the present technology include other features than those described herein. For example, one or two leads 111 can be positioned generally end-to-end, with at least a portion overlapping, or without overlapping, at or near the patient's midline, and can span vertebral levels from about T1 to about T12, or about T5 to about T12. In some embodiments, a first lead 111 is positioned at or near the patient's midline at a first position within T5 to T8 and a second lead 111 is positioned at or near the patient's midline at a second position from T9 to T12. Without intending to be bound by any particular theory, positioning one or more leads 111 at the patient's midline is thought to address ADHF by stimulating one or more sympathetic nerves to treat ADHF, and/or one or more symptoms associated with ADHF, and/or pain, and/or sympathetic dysfunction. The devices and systems of the present technology can include more than one internal stimulator and/or more than one external stimulator that can be configured for wireless stimulation, such as by using electromagnetic waves.

Several aspects of the technology are embodied in computing devices, e.g., programmed/programmable pulse generators, controllers and/or other devices. The computing devices on/in which the described technology can be implemented may include one or more central processing units, memory, input devices (e.g., input ports), output devices (e.g., display devices), storage devices, and network devices (e.g., network interfaces). The memory and storage devices are computer-readable media that may store instructions that implement the technology. In some embodiments, the computer readable media are tangible media. In some embodiments, the data structures and message structures may be stored or transmitted via an intangible data transmission medium, such as a signal on a communications link, Various suitable communications links may be used, including but not limited to a local area network and/or a wide-area network.

In some embodiments, systems configured in accordance with the present technology to treat ADHF include an implantable electrical signal generator having a computer-readable storage medium, and an implantable signal delivery element coupled to the signal generator and configured to be positioned at or proximate to the patient's spinal cord at the target location and configured to apply thetherapy signal to the target location. In addition, the computer-readable storage medium has instructions that, when executed, determine (1) activity of one or more sympathetic nerves, (2) a sympathetic activity level indicator that is or includes the patient's splanchnic nerve activity, (3) an activity of the renal nerve, (4) the patient's cardiac sympathetic outflow, or any combination of (1)-(4); and adjust the signal applied by the signal delivery element in response to any one or combination of (1)-(4).

Systems configured in accordance with the present technology can further include a sensor in communication with the computer-readable storage medium, wherein the sensor is configured to detect the activity of the one or more sympathetic nerves of the patient, and wherein the instructions, when executed, calculate the patient's sympathetic activity level. In response to a determined sympathetic activity level indicator different from a predetermined target threshold, the system (1) ceases to apply the electrical signal, (2) starts application of the electrical signal, and/or (3) increases and/or decreases at least one of a frequency, an amplitude, or a pulse width of the electrical signal. The determined sympathetic activity level indicator and/or the predetermined target threshold can be generally similar or different when detected at a first target location and second target location. For example, the first target location can be a first sympathetic nerve associated with the patient's heart having a first sympathetic activity level indicator that is greater than a second sympathetic activity level indicator of the patient's lung and associated with a second sympathetic nerve at the second target location. In this example, the system can start application of the electrical signal to the first target location and cease application of the electrical signal to the second target location. In other examples, the system can perform one or more of options (1), (2), or (3) at any target location to achieve a desired outcome of treating the patient having ADHF.

In some embodiments, it is important that the signal delivery device 110 and in particular, the therapy or electrical contacts C of the device, be placed at or proximate to a target location that is expected (e.g., by a practitioner) to produce efficacious results in the patient when the device 110 is activated. Section 4.0 describes techniques and systems for positioning leads 111 in the patient's spinal column to deliver neural modulation signals to treat the patient's ADHF and/or one or more symptoms associated with ADHF.

### 4.0 REPRESENTATIVE METHODS FOR TREATING ACUTE DECOMPENSATED HEART FAILURE

The autonomic nervous system (ANS) is largely responsible for automatically and subconsciously regulating many systems of the body, including the cardiovascular, renal, gastrointestinal, and thermoregulatory systems. By regulating these systems, the ANS can enable the body to adapt to changes in the environment. Autonomic nerve fibers innervate a variety of tissues, including cardiac muscle, smooth muscle, lungs, and glands. These nerve fibers help to regulate functions corresponding to the foregoing tissues, including but not limited to blood pressure, blood flow, bronchial dilation, gastric dysmotility, and glandular secretions. The autonomic nervous system includes the sympathetic system and the parasympathetic system, and it is thought that the sympathetic system can be modulated to treat ADHF and/or one or more symptoms associated with ADHF as described above in Section 2.0. Additional features of the ANS and application of therapeutic modulation signals to modulate a patient's ANS are described in U.S. Pat. No. 9,833,614, incorporated by reference herein in its entirety.

Without intending to be bound by any particular theory, it is believed that ADHF may be caused, at least in part, by altered effects of the patient's sympathetic system. One approach to treating ADHF in accordance with some embodiments of the present technology is to apply therapeutic signals to the patient's spinal column to modulate one or more effects of the patient's sympathetic system, such as those described in Section 2.0. One possible mechanism of action by which therapeutic signals are expected to treat ADHF, and/or one or more symptoms associated with ADHF, is to modulate the patient's sympathetic nervous system, such as increasing, decreasing, and/or inhibiting at least a portion of its activity. It is believed that therapeutic signals can operate in a manner similar and/or analogous to that corresponding to pain treatment to modulate at least a portion of the patient's sympathetic system. The effect of therapeutic modulation signals on wide dynamic range (WDR) neurons described previously with respect to pain is thought to apply similarly to ADHF. These effects on WDR neurons are described in U.S. Pat. No. 9,833,614, previously incorporated by reference herein in its entirety. Methods and systems for treating the patient's ADHF, one or more symptoms associated with ADHF, and/or pain, by applying therapeutic modulation signals to thoracic neural populations, are discussed immediately below.

It is believed that therapeutic modulation at or near one or more of the patient's thoracic vertebrae T1 to T12, and in particular at T5 to T12, can treat the patient's ADHF, one or more symptoms associated with ADHF, and/or pain, without paresthesia, without adverse sensory or motor effects, and/or in a manner that persists after the modulation ceases. In some embodiments, the present technology includes methods for treating a patient's ADHF by positioning an implantable signal delivery device proximate to a target location at or near the patient's spinal cord within a vertebral range of about T1 to about T12, directing an electrical therapy signal having a frequency of 1.2 kHz to 100 kHz to the target location via the implantable signal delivery device, and modulating one or more of the patient's sympathetic nerves. For example, the electrical therapy signal has a frequency of about 10 kHz, a pulse width of about 20 microseconds to about 175 microseconds, and/or an amplitude from about 20% of the patient's sensory threshold to about 90% of the patient's sensory threshold (e.g., from about 0.1 mA to about 20 mA). During and following application, the electrical signal modulates the patient's splanchnic nerve activity to treat the patient's ADHF. For example, the one or more sympathetic nerves are sympathetic nerves that are associated with the patient's circulation, such as the greater splanchnic nerve, the lesser splanchnic nerve, the least splanchnic nerve, and/or the renal nerve.

In some embodiments, the patient's sympathetic activity is determined by monitoring one or more physiologic parameters, such as, acute heart rate, chronic heart rate, lung congestion, edema, splanchnic circulation, cardiac sympathetic outflow, and sympathetic nervous system output (e.g., using an electrodermal sensor and/or one or more heart rate variability components).

FIG. 2 is a partially schematic illustration of a patient's sympathetic nervous system, including the brain 210 and spinal column 230, and the organs innervated by the corresponding sympathetic nerves 215 (based generally on information from Martini, Ober, and Nath, “Visual Anatomy and Physiology,” 2011 (published by Pearson Education)). Without intending to be bound by any particular theory, it is thought that applying a therapeutic modulation signal to a target location at or between T5 to T12 of the patient's thoracic region modulates one or more of the patient's sympathetic nerves 215, such as the splanchnic nerve, the greater splanchnic nerve, the lesser splanchnic nerve, and the least splanchnic nerve. In some embodiments, the electrical therapy signal modulates one or more of the sympathetic nerves 215 innervating the patient's heart 261 and/or lung 263. By modulating one or more of the sympathetic nerves 215, it is thought that the electrical signal prevents and/or decreases one or more symptoms and/or conditions associated with ADHF, thereby treating the patient's ADHF.

A lead body 111 (shown prior to implant in FIG. 2) can be positioned at or proximate to the thoracic region 220 (e.g., T1-T12) of the patient's spinal column 230. While not shown in FIG. 2, the lead body 111 can be a first implantable signal delivery device that includes a first plurality of contacts and can be positioned on a first side of a midline of the patient's spinal column 230. In some embodiments, a second implantable signal delivery device (not shown) having a second plurality of contacts C can be positioned on a second side of the midline. For example, the first plurality of contacts C can be positioned longitudinally along the first side of the midline (e.g., from T5 to T8), and the second plurality of contacts C can be positioned longitudinally along the second side of the midline (e.g., from T9 to T12). In some embodiments, the first implantable signal delivery device and the second implantable signal delivery device can be positioned on the same side of the midline (e.g., either the first side or the second side). In some embodiments, the vertebral ranges in which the first plurality of C and the second plurality of contacts C can differ from those disclosed herein.

In some embodiments, the first implantable signal delivery device and the second implantable signal delivery device overlap by about ½ to about ⅓ of a length of each of the signal delivery devices. However, in some embodiments, the first implantable signal delivery device and the second implantable signal delivery device do not overlap. For example, the second implantable signal delivery device can be positioned at least generally end to end such that the first plurality of contacts C and the second plurality of contacts C extend generally longitudinally along the midline from T5 to T12.

After positioning, the therapeutic modulation signal can be delivered to the patient's target location at generally the same time (e.g., simultaneously or approximately simultaneously) via two or more implantable therapeutic signal delivery devices. In general, modulating the sympathetic nerves to treat ADHF and/or one or more symptoms associated with ADHF may be achieved following delivery of one or more therapeutic modulation signals having one or more stimulation parameters at or proximate to one or more of T1 to T12 vertebrae, e.g., T5 to T12. For example, the stimulation parameters include, but are not limited to, amplitude, frequency, pulse width, duty cycling, and whether stimulation is applied at or proximate to the patient's left side and/or the patient's right side of their midline.

In some embodiments, one or more therapeutic modulation signals can be delivered to a target location proximate to or at one or more of T2 to T4, and T5 to T12. When delivered to the target location proximate to or at one or more of T5 to T12, these therapeutic modulation signals are thought to modulate at least a portion of the patient's sympathetic nerves that innervate the patient's gastrointestinal tract and splanchnic circulation. When delivered to the target location proximate to or at one or more of T2 to T4, these therapeutic modulation signals are thought to modulate at least a portion of the patient's sympathetic nerves that innervate the patient's heart and lungs. Modulation of the patient's sympathetic nerves corresponding to one or more of the target locations at or proximate to T5 to T12 to treat the patient's ADHF can be achieved by positioning one or more implantable therapeutic signal delivery devices at or proximate to one or more of T5 to T12. In some embodiments, positioning one or more implantable therapeutic signal delivery devices may further include combination placement (e.g., spanning more than one thoracic region such as T2-T4 and/or T5-T7) rather than at individually at T1, T2, T3, or T4, and so on. In addition, the modulation may, in some instances, be directed to the splanchnic nerve itself and/or nerves associated with the splanchnic nerve. In any of these embodiments, the technology can include chronic stimulation and/or intermittent stimulation.

FIG. 3 is a flow diagram illustrating a method for treating ADHF in accordance with embodiments of the present technology. Method 300 includes positioning implantable signal delivery devices as shown in FIGS. 1A and 1B (e.g., a lead, paddle or other suitable device) proximate to a target location at or near the patient's spinal cord within a vertebral range of about T1 to about T12 (block 302). The spinal cord region can include epidural and/or subdural regions, at or off the midline, including the dorsal root, dorsal root entry zone and the dorsal root ganglia. The particular location within the spinal cord region can depend upon the patient and/or the particular embodiment of the present technology. For example, one device may be implanted on one side of the spinal cord midline 189 (FIG. 1), and a second device may be implanted on the other side of the spinal cord midline 189. In another example, one device may be implanted in the epidural space and another device may be implanted subcutaneously. The leads 111 (FIGS. 1A and 1B) may be positioned just off the spinal cord midline 189 (e.g., about 1 mm offset) in opposing lateral directions so that the two leads 111 are spaced apart from each other by about 2 mm. The leads 111 may be implanted at a vertebral level ranging from, for example, about T1 to about T12 to treat ADHF, one or more symptoms associated with ADHF, and/or pain. In some embodiments, the leads 111 can be implanted at other vertebral levels to address other patient indications.

The method 300 further includes directing an electrical therapy signal to the target location via the implantable signal delivery device 111, wherein the electrical signal has a frequency in a frequency range of from 1.2 kHz to 100 kHz (block 304). The electrical contacts C are activated with (e.g., have applied to them) one or more therapy signals in accordance with some embodiments of the present technology, in order to modulate one or more of the patient's sympathetic nerves to treat the patient's ADHF, one or more symptoms associated with ADHF, and/or pain (block 306).

In some embodiments, methods for treating a patient having ADHF include monitoring at least one physiological parameter of the patient, automatically detecting a change in at least one of the physiological parameters that is outside of a predetermined threshold (e.g., above or below) and indicates increased sympathetic nervous system activity, and based on the detected parameter, delivering an electrical signal to the patient's spinal cord via at least one signal delivery element positioned in the patient's epidural space. In addition, in some embodiments, methods for treating a patient having ADHF include monitoring the patient's ADHF by determining the patient's sympathetic activity, and in response to monitoring results, adjusting at least one signal delivery parameter in accordance with which the electrical signal is applied to the target location, such as frequency, amplitude, pulse width, duty cycle, and normal slow wave frequency, or terminating delivery of the electrical therapy signal.

While embodiments of the present technology may create some effect on normal motor and/or sensory signals, the effect is below a level that the patient can reliably detect intrinsically, e.g., without the aid of external assistance via instruments or other devices, Accordingly, the patient's levels of motor signaling and other sensory signaling (other than signaling associated with ADHF) can be maintained at pre-treatment levels. For example, the patient can experience a significant reduction in ADHF and/or one or more associated symptoms, largely independent of the patient's movement and position. In particular, the patient can assume a variety of positions, consume various amounts of food and liquid, and/or undertake a variety of movements associated with activities of daily living and/or other activities, without the need to adjust the parameters in accordance with which the therapy is applied to the patient (e.g., the signal amplitude). This result can greatly simplify the patient's life and reduce the effort required by the patient to undergo ADHF treatment (or treatment for associated symptoms) while engaging in a variety of activities. This result can also provide an improved lifestyle for patients who experience symptoms associated with ADHF during sleep. In some embodiments, the therapeutic signal is delivered to patients continuously or intermittently, such as at various times throughout the day.

In some embodiments, patients can choose from a number of signal delivery programs (e.g., two, three, four, five, or six), each with a different amplitude and/or other signal delivery parameter, to treat the patient's ADHF. In some embodiments, the patient activates one program before sleeping and another after waking, or the patient activates one program before sleeping, a second program after waking, and a third program before engaging in particular activities that would otherwise trigger, enhance, or otherwise exacerbate the patient's ADHF, such as pre-prandial, prandial, and/or post-prandial activities, and/or pre-exercise, exercise, and/or post-exercise related activities.

In some embodiments, one or more of the programs are generally the same. This reduced set of patient options can greatly simplify the patients ability to easily manage ADHF, without reducing (and in fact, increasing) the circumstances under which the therapy effectively addresses ADHF. In some embodiments which include multiple programs, the patient's workload can be further reduced by automatically detecting a change in patient circumstance, and automatically identifying and delivering the appropriate therapy regimen. Additional details of such techniques and associated systems are disclosed in U.S. Pat. No. 8,355,797, incorporated herein by reference.

In some embodiments, rather than the patient activating one or more programs, the systems, devices, and methods described herein automatically detect a beginning and/or an end of one or more prandial events. For example, the systems, devices, and methods described herein can automatically detect one or more events that exacerbate the patient's ADHF and/or a symptom associated with ADHF, by monitoring the patient's sympathetic nervous system using one or more of the techniques described herein, either intermittently or continuously, and if a change in the patient's sympathetic nervous system is detected, the systems, devices, and methods described herein can automatically deliver a therapy signal. In some embodiments, the systems, devices, and methods described herein can include one or more sensors configured to monitor the patient's sympathetic nervous system by detecting changes in one or more organs and/or tissues modulated by the sympathetic nervous system.

In some embodiments, electrical stimulation may be administered on a pre-determined schedule or on an as-needed basis. Administration may continue for a pre-determined amount of time, or it may continue indefinitely until a specific therapeutic benchmark is reached, for example, until an acceptable reduction in one or more symptoms. In some embodiments, electrical stimulation may be administered one or more times per day, one or more times per week, once a week, once a month, or once every several months. Since electrical stimulation is thought to improve ADHF and/or symptoms associated with ADHF over time with repeated use, the patient is expected to need less frequent electrical stimulation therapy. In some embodiments, the therapy can be delivered when the patient's ADHF recurs or increases in severity. Administration frequency may also change over the course of treatment. For example, a patient may receive less frequent administrations over the course of treatment as certain therapeutic benchmarks are met. The duration of each administration (e.g., the actual time during which a subject is receiving electrical stimulation) may remain constant throughout the course of treatment, or it may vary depending on factors such as patient health, internal pathophysiological measures, or symptom severity. In some embodiments, the duration of each administration may range from 1 to 4 hours, 4 to 12 hours, 12 to 24 hours, 1 day to 4 days, or 4 days or greater.

As described above, the therapeutic modulation signal can have an amplitude in an amplitude range of between about 20% to about 90% of the patient's sensory threshold, at a frequency of about 10 kHz, and/or about a 30 microsecond pulse width and can be applied at a particular vertebral level corresponding to the patient's heart, lung, organ, and/or tissue of interest, such as at the thoracic vertebral levels (e.g., T1 to T12) to modulate activity of the patient's sympathetic nervous system (e.g., the sympathetic nerves innervating or otherwise corresponding to the patient's heart and lungs, gastrointestinal tract including the patient's stomach, and liver, and/or bladder), and/or any other organs and/or tissues having sympathetic innervation or which may be affected by an organ or tissue having sympathetic innervation. Further details of particular vertebral levels and associated organs are described herein and in U.S. Pat. No. 8,170,675, previously incorporated herein by reference. In some embodiments, additional stimulation parameters can be applied to one or more of these vertebral levels to treat ADHF and/or one or more associated symptoms of ADHF, such as those described below.

In some embodiments, therapeutic electrical stimulation to treat ADHF, and/or one or more symptoms associated with ADHF, is performed with at least a portion of the therapy signal at amplitudes within amplitude ranges of: about 0.1 mA to about 20 mA; about 0.5 mA to about 10 mA; about 0.5 mA to about 7 mA; about 0.5 mA to about 5 mA; about 0.5 mA to about 4 mA; about 0.5 mA to about 2.5 mA; and in some embodiments, surprisingly effective results have been found when treating certain medical conditions with amplitudes below 7 mA.

In some embodiments, therapeutic electrical stimulation to treat ADHF, and/or one or more symptoms associated with ADHF, is performed with at least a portion of the therapy signal having a pulse width in a pulse width range of from about 10 microseconds to about 333 microseconds; from about 10 microseconds to about 166 microseconds; from about 25 microseconds to about 166 microseconds; from about 25 microseconds to about 100 microseconds; from about 30 microseconds to about 100 microseconds; from about 33 microseconds to about 100 microseconds; from about 50 microseconds to about 166 microseconds; and in some embodiments, surprisingly effective results have been found when treating certain medical conditions with pulse widths from about 25 microseconds to about 100 microseconds; and from about 30 microseconds to about 40 microseconds.

In some embodiments, therapeutic electrical stimulation to treat ADHF, and/or one or more symptoms associated with ADHF, is performed with at least a portion of the therapy signal having a 30 microsecond cathodic pulse followed by a 20 microsecond interphase delay followed by a 30 microsecond anodic pulse followed by another 20 microsecond interphase delay. The total phase time duration of 100 microseconds corresponds to a frequency of 10 kHz. The total phase time duration may range from 10 to 833 microseconds corresponding to frequencies ranging from 1200 Hz to 100 kHz. In some embodiments, the interphase delays may differ from 20 microseconds and may range from 0 to 833 microseconds. In some embodiments, the cathodic pulse may differ from 30 microseconds and may range from 0 to 833 microseconds. In some embodiments, the anodic pulse may differ from 30 microseconds and may range from 0 to 833 microseconds.

Aspects of the therapy provided to the patient may be varied, while still obtaining beneficial results. For example, the amplitude of the applied signal can be ramped up and/or down and/or the amplitude can be increased or set at an initial level to establish a therapeutic effect, and then reduced to a lower level to save power without forsaking efficacy, as is disclosed in U.S. Patent Publication No. 2009/0204173, incorporated herein by reference. The signal amplitude may refer to the electrical current level, e.g., for current-controlled systems or to the electrical voltage level, e.g., for voltage-controlled systems. The specific values selected for the foregoing parameters may vary from patient to patient and/or from indication to indication and/or on the basis of the selected electrical stimulation location, such as the sacral region. In addition, the present technology may make use of other parameters, in addition to or in lieu of those described above, to monitor and/or control patient therapy. For example, in cases for which the pulse generator includes a constant voltage arrangement rather than a constant current arrangement, the current values described above may be replaced with corresponding voltage values.

In some embodiments, the parameters in accordance with which the pulse generator provides signals can be modulated during portions of the therapy regimen. For example, the frequency, amplitude, pulse width and/or signal delivery location can be modulated in accordance with a preset program, patient and/or physician inputs, and/or in a random or pseudorandom manner. Such parameter variations can be used to address a number of potential clinical situations, including changes in the patients perception of one or more symptoms corresponding to the condition being treated, changes in the preferred target neural population, and/or patient accommodation or habituation.

In some embodiments, a practitioner can obtain feedback from the patient to detect whether the patient has, and/or is experiencing, ADHF, one or more risk factors related to ADHF, and/or one or more symptoms associated with ADHF, and/or the effect of the therapeutic modulation signal on any of the foregoing conditions and/or symptoms. Monitoring a patient's ADHF and/or symptoms associated with ADHF can be performed on a continuous basis using one or more sensing elements (referred to herein as a “sensing element”) for detecting neural signals and/or neural responses of the patient before, during and/or after the application of electrical stimulation signals to the patient. In some embodiments, the sensing element can be carried by the signal generator 101, the signal delivery elements 110, and/or other implanted components of the system 100, as previously described with reference to FIG. 1A. As such, the sensing element may be positionable in an area proximate to the target treatment site where electrical stimulation is being delivered. In some embodiments, the sensing element can be positioned separate from the signal generator 101 and/or the signal delivery elements 104. For example, the sensing elements may be implanted in an area separate from the area where electrical stimulation is being delivered, or in an extracorporeal manner. When separated from one another, the sensing element and the signal generator 101 may be wirelessly coupled to one another (e.g., via a Bluetooth link).

Representative sensing elements can include an impedance sensor, a chemical sensor, a biosensor, an electrochemical sensor, a hemodynamic sensor, an optical sensor and/or other suitable implantable sensing devices. The sensing element can detect one or more neural signal(s) and/or neural response(s) (e.g., electrical signals corresponding to action potentials) from the nerve or neural population, and the system (e.g., the system 100 referenced in FIG. 1A) can use the detected neural signal(s) and/or neural response(s) to identify the patient's hemodynamic state at a particular moment in time. The neural response(s) can be detected frequently enough such that an upward or downward trend of the data corresponding to the patient's hemodynamic state can be determined, or at least estimated.

The detected neural signal(s) and/or response(s) can include characteristics that may be measured and used to identify the patient's hemodynamic state at a particular point in time. Characteristics can include, for example, signal strength (e.g., whether a value of the signal is above a pre-determined threshold value), frequency (e.g., number of action potentials fired in a given time), amplitude and/or velocity, amongst other measurable characteristics. In some embodiments, changes of a characteristic from one or more previous neural signals or neural responses, and/or rates of change of a characteristic from previous neural signals or neural responses, can be used in a similar manner. Measurements corresponding to the characteristics can then be used to identify the patient's hemodynamic state at a particular moment in time, and risk for developing and/or experiencing ADHF, and/or one or more symptoms associated with ADHF. In some embodiments, the identified hemodynamic state may be determined or estimated based on a pre-determined correlation between the values of the characteristics of the neural response(s) and the hemodynamic state of the patient or a similarly situated patient.

Monitoring the patient's hemodynamic state, as disclosed herein, can be performed in tandem with modulating electrical therapy signals to the patient. Stated otherwise, the patient's hemodynamic state can be continuously (or periodically) monitored using the methods described herein, and used to determine or adjust the signal delivery parameters so as to improve the effect of the modulated electrical therapy signals, For example, the practitioner can (a) continuously observe/monitor the patient's hemodynamic state to determine a baseline level, (b) direct an electrical therapy signal (e.g., a signal having a frequency from 1.2 kHz to 100 kHz) to a neural population of the patient via an implantable signal delivery device, (c) monitor the patient's hemodynamic state after directing the therapy signal to report changes in ADHF and/or one or more associated symptoms, and/or other functions, and (d) if necessary, adjust the electrical therapy signal to achieve a more desirable hemodynamic state. Adjusting the electrical therapy signal can include adjusting one or more signal delivery parameters (e.g., frequency, amplitude, pulse width, duty cycle, and normal slow wave frequency) of the subsequent electrical signal to be applied to the target location. Steps (a)-(d) can be performed iteratively to improve or achieve a desired result for the patient. Suitable methods and products for monitoring this system include those where the patient's response to the electrical stimulation therapy can be adjusted. Additionally, the monitoring methods of the present technology can be used as a diagnostic tool to pre-emptively monitor progression and/or onset of ADHF and/or one or more associated symptoms. For example, the implantable sensing device can be implanted prior to an onset of ADHF, and data from the sensing device can be used to identify trends that may be used to suggest the onset of ADHF. Data from the sensing device can be wirelessly transmitted (e,g., to a server) such that the practitioner can remotely monitor the data and identify trends.

The therapeutic modulation signal can operate on the targeted organ or organs in accordance with any of a number of mechanisms. For example, the therapeutic modulation signal can have an effect on a network of neurons, rather than an effect on a particular neuron. This network effect can in turn operate to reduce and/or otherwise inhibit one or more effects of the sympathetic nervous system described above. The foregoing mechanisms of action can have a cascading effect on other systems. For example, the effect of inhibiting the sympathetic nervous system can be indirect. It is believed that, as a result of this indirect effect, the ultimate effect on the organ may not occur instantaneously, but rather may take time (e.g., days) to develop, in response to a modulation signal that is applied to the patient for over a similar period of time (e.g., days).

A variety of suitable devices for administering an electrical signal to the T1-T12 region are described in greater detail above in Section 3.0 and may also be described in the references incorporated by reference herein. Examples of devices for administering an electrical signal that can treat ADHF and/or one or more associated symptoms are disclosed in U.S. Pat. No. 8,694,108 (66245-8024.US00) and U.S. Pat. No. 8,355,797 (66245-8012.US02), both of which are incorporated herein by reference in their entireties, and attached as Appendices H and D. For example, applying electrical stimulation can be carried out using suitable devices and programming modules specifically programmed to carry out any of the methods described herein. For example, the device can comprise a lead, wherein the lead in turn comprises an electrode. In some embodiments, administration of electrical stimulation comprises a positioning step (e.g., placing the lead such that an electrode is in proximity to the T1 to T12 region) and a stimulation step (e.g., transmitting an electrical signal (i.e., therapy signal) through the electrode), In some embodiments, a device that is used for applying an electrical signal to the spinal cord may be repurposed with or without modifications to administer an electrical signal to another target tissue or organ, e.g., a T1-T12 region, a cortical, sub-cortical, intra-cortical, or peripheral target. As such, any of the herein described systems, sub-systems, and/or sub-components serve as means for performing any of the herein described methods.

Many of the embodiments described above were described in the context of treating ADHF with modulation signals applied to the T1 to T12 vertebral levels. T2D represents an example indication that is expected to be treatable with modulation applied at this location. In some embodiments, modulation signals having parameters (e.g., frequency, pulse width, amplitude, and/or duty cycle) generally similar to those described above can be applied to other patient locations, to address other indications.

The methods disclosed herein include and encompass, in addition to methods of making and using the disclosed devices and systems, methods of instructing others to make and use the disclosed devices and systems. For example, a method in accordance with a particular embodiment includes treating ADHF by applying an electrical signal to the patient's T1 to T12 region, with the electrical signal having a frequency in a range of from about 1.5 kHz to about 100 kHz, a pulse width in a pulse width range of 33 microseconds to 333 microseconds and an amplitude in an amplitude range of 0.1 mA to 20 mA, and/or a duty cycle of 5% to 75%.

A method in accordance with another embodiment includes programming a device and/or system to deliver such a method, instructing or directing such a method. Accordingly, any and all methods of use and manufacture disclosed herein also fully disclose and enable corresponding methods of instructing such methods of use and manufacture.

From the foregoing, it will be appreciated that some embodiments of the present technology have been described herein for purposes of illustration, but that various modifications may be made without deviating from the technology. As described above, signals having the foregoing characteristics are expected to provide therapeutic benefits for patients having ADHF, when stimulation is applied at T1 to T12. In some embodiments, the present technology can be used to address one or more pain indications, such as those described in the references incorporated by reference, besides and/or in addition to ADHF and/or one or more associated symptoms.

The methods, systems, and devices described above may, in addition to treating ADHF, be used to deliver a number of suitable therapies, e.g., paresthesia-based therapies and/or paresthesia-free therapies, for patients experiencing pain and/or diseases or conditions other than ADHF. Examples of such therapies and associated methods, systems, and devices are described in U.S. Patent Publication Nos. 2009/0204173 and 2010/0274314, the respective disclosures of which are herein incorporated by reference in their entireties, and attached as Appendices G and I.

### 5.0 ADDITIONAL EMBODIMENTS

The methods, systems, and devices described above may be used to deliver a number of suitable therapies, e.g., paresthesia-based therapies and/or paresthesia-free therapies. Examples of such therapies and associated methods, systems, and devices are described in U.S. Patent Publication Nos. 2009/0204173 and 2010/0274314, the respective disclosures of which are herein incorporated by reference in their entireties.

Certain aspects of the technology described in the context of particular embodiments may be combined or eliminated in other embodiments. For example, many of the embodiments described above refer to delivery of the electrical therapy signal using two or more leads. In some embodiments, the electrical therapy signals described herein can be delivered with one lead, or more than one lead, and includes the leads described herein and those described in the references incorporated herein. In addition, while advantages corresponding to some embodiments of the technology have been described in the context of some embodiments, other embodiments may also exhibit such advantages, and not all embodiments need necessarily exhibit such advantages to all within the scope of the present technology. Accordingly, the disclosure and associated technology can encompass other embodiments not expressly shown or described herein.

As used herein, the term “and/or” when used in the phrase “a and/or b” refers to a alone, to b alone, and to both a and b. A similar manner of interpretation applies to the term “and/or” when used in a list of more than two terms.

To the extent any materials incorporated by reference herein conflict with the present disclosure, the present disclosure controls.

### 6.0 REPRESENTATIVE EXAMPLES

The following examples are provided to further illustrate embodiments of the present technology and are not to be interpreted as limiting the scope of the present technology. To the extent that certain embodiments or features thereof are mentioned, they are merely for purposes of illustration and, unless otherwise specified, are not intended to limit the present technology. One skilled in the art may develop equivalent means without the exercise of inventive capacity and without departing from the scope of the present technology. It will be understood that many variations can be made in the procedures herein described while still remaining within the bounds of the present technology. Such variations are intended to be included within the scope of the presently disclosed technology. As such, embodiments of the presently disclosed technology are described in the following representative examples.
1. A method for treating a patient having acute decompensated heart failure (ADHF), comprising:
positioning an implantable signal delivery device proximate to a target location at or near the patient's spinal cord within a vertebral range of about T1 to about T12;
directing an electrical therapy signal to the target location via the implantable signal delivery device, wherein the electrical signal has a frequency in a frequency range of from 1.2 kHz to 100 kHz to modulate one or more of the patient's sympathetic nerves and treat the patient's ADHF,
2. The method of example 1 wherein the electrical signal does not create paresthesia in the patient.
3. The method of example 1 or 2 wherein the vertebral range is about T5 to about T12.
4. The method of any of the preceding examples wherein the electrical signal modulates the patient's splanchnic nerve activity.
5. The method of any of the preceding examples wherein modulation of the patient's splanchnic nerve activity prevents and/or decreases venous congestion and/or pulmonary congestion.
6. The method of any of the preceding examples wherein modulation of the patient's splanchnic nerve activity prevents and/or decreases fluid retention.
7. The method of any of the preceding examples wherein modulation of the patient's splanchnic nerve activity increases the patient's splanchnic circulation.
8. The method of any of the preceding examples wherein modulation of the patient's splanchnic nerve activity prevents and/or decreases lung congestion.
9. The method of any of the preceding examples wherein modulation of the patient's splanchnic nerve activity prevents and/or decreases edema.
10. The method of any of the preceding examples wherein modulation of the patient's splanchnic nerve activity reduces activity of the patient's sympathetic nervous system.
11. The method of any of the preceding examples wherein the electrical signal further treats the patient's pain.
12. The method of any of the preceding examples wherein the implantable signal delivery device is positioned in the patient's epidural space.
13. The method of any of the preceding examples, further comprising positioning a second implantable signal delivery device proximate to the target location.
14. The method of any of the preceding examples wherein the implantable signal delivery device is a paddle lead.
15. The method of any of the preceding examples wherein the electrical therapy signal has a frequency of about 10 kHz.
16. The method of any of the preceding examples wherein the electrical therapy signal has a pulse width of about 20 microseconds to about 175 microseconds.
17. The method of any of the preceding examples wherein the electrical therapy signal has an amplitude from about 20% of the patient's sensory threshold to about 90% of the patient's sensory threshold.
18. The method of any of the preceding examples wherein the electrical therapy signal has an amplitude of from about 0.1 mA to about 20 mA.
19. The method of any of the preceding examples wherein the one or more sympathetic nerves are sympathetic nerves that associated with the patient's circulation.
20. The method of any of the preceding examples wherein the one or more sympathetic nerves are selected from the group consisting of the greater splanchnic nerve, the lesser splanchnic nerve, and the least splanchnic nerve.
21. The method of any of the preceding examples wherein modulating the one or more sympathetic nerves reduces mobilization of venous reservoirs, reduces splanchnic congestion, and/or reduces the patient's effective circulatory volume.
22. The method of any of the preceding examples wherein splanchnic congestion is reduced by reducing the patient's retention of sodium and/or fluid.
23. The method of any of the preceding examples, further comprising:
monitoring the patient's ADHF by determining the patient's sympathetic activity; and
in response to results obtained from monitoring the patient's ADHF;
   adjusting at least one signal delivery parameter in accordance with which the electrical signal is applied to the target location, wherein the signal delivery parameter is selected from the group consisting of frequency, amplitude, pulse width, duty cycle, and normal slow wave frequency, or
   terminating delivery of the electrical therapy signal.
24. The method of any of the preceding examples wherein the patient's sympathetic activity is determined by monitoring one or more physiologic parameters selected from the group consisting of acute heart rate, chronic heart rate, lung congestion, edema, splanchnic circulation, and sympathetic nervous system output.
25. The method of any of the preceding examples wherein the patient's sympathetic nervous system output is monitored using an electrodermal sensor and/or one or more heart rate variability components.
26. A method for treating a patient having acute decompensated heart failure (ADHF), comprising:
monitoring at least one physiological parameter of the patient;
automatically detecting a change in the at least one physiological parameter that is outside of a predetermined threshold, wherein the change in the at least one physiological parameter is indicative of increased sympathetic nervous system activity; and
based on the detected parameter, delivering an electrical signal to the patient's spinal cord via at least one signal delivery element positioned in the patient's epidural space at a thoracic vertebral level from about T1 to about T12, the electrical signal having a frequency of from about 1 kHz to about 100 kHz.
27. The method of example 26 wherein the thoracic vertebral is from about T5 to about T12.
28. The method of example 26 or example 27 wherein the electrical signal does not create paresthesia in the patient.
29. The method of any one of examples 26-28 further comprising delivering the electrical signal to the patient's spinal cord via at least one signal delivery element positioned at the thoracic vertebral level.
30. The method of any one of examples 26-29 wherein the electrical therapy signal has a frequency of about 10 kHz to about 50 kHz.
31. The method of any one of examples 26-30 wherein the electrical therapy signal has a pulse width of about 2 microseconds to about 175 microseconds.
32. The method of any one of examples 26-31 wherein the electrical therapy signal has an amplitude from about 20% of the patient's sensory threshold to about 90% of the patient's sensory threshold.
33. The method of any one of examples 26-32 wherein the electrical therapy signal has an amplitude of from about 0.1 mA to about 20 mA.
34. The method of any one of examples 26-33 wherein the one or more sympathetic nerves are sympathetic nerves associated with the patient's circulation.
35. The method of any one of examples 26-34 wherein the one or more sympathetic nerves are selected from the group consisting of the greater splanchnic nerve, the lesser splanchnic nerve, and the least splanchnic nerve.
36. The method of any one of examples 26-36 wherein modulating the one or more sympathetic nerves reduces mobilization of venous reservoirs, reduces splanchnic congestion, and/or reduces the patient's effective circulatory volume.
37. The method of any one of examples 26-36 wherein splanchnic congestion is reduced by reducing the patient's retention of sodium and/or fluid.
38. The method of any one of examples 26-37 wherein the patient's sympathetic activity is determined by monitoring one or more physiologic parameters selected from the group consisting of acute heart rate, chronic heart rate, lung congestion, edema, splanchnic circulation, and sympathetic nervous system output.
39. The method of any one of examples 26-38 wherein the patient's sympathetic nervous system output is monitored using an electrodermal sensor and/or one or more heart rate variability components.
40. The method of any one of examples 26-39 wherein the electrical signal further treats the patient's pain.
41. The method of any one of examples 26-40 wherein the electrical signal further treats the patient's ADHF.
42. A system for treating acute decompensated heart failure (ADHF) in a patient, comprising:
an implantable electrical signal generator having a computer readable storage medium;
an implantable signal delivery element coupled to the signal generator, wherein the signal delivery element is configured to be positioned at or proximate to the patient's spinal cord at a target location from about T1 to about T12, and wherein the signal delivery element is configured to apply about 1 kHz to about 100 kHz to the target location; and
wherein the computer-readable storage medium has instructions that when executed:
determine activity of one or more sympathetic nerves; and
adjust the signal applied by the signal delivery element in response to the determined sympathetic nerve activity.
43. The system of example 42, further comprising a sensor in communication with the computer-readable storage medium, wherein the sensor is configured to detect the activity of the one or more sympathetic nerves of the patient, and wherein the instructions, when executed, calculate the patient's sympathetic activity level.
44. The system of example 42 or example 43 wherein the instructions, when executed, and in response to a determined sympathetic activity level indicator greater than or equal to a predetermined target threshold, cease to apply the electrical signal.
45. The system of any one of examples 42-44 wherein the instructions, when executed, and in response to a determined sympathetic activity level indicator less than or equal to a predetermined target threshold, start application of the electrical signal.
46. The system of any one of examples 42-45 wherein the instructions, when executed, and in response to a determined sympathetic activity level indicator less than or equal to a predetermined target threshold, increase at least one of a frequency, an amplitude, or a pulse width of the electrical signal.
47. The method of any one of examples 42-46 wherein the patient's sympathetic activity is determined by monitoring one or more physiologic parameters selected from the group consisting of acute heart rate, chronic heart rate, lung congestion, edema, splanchnic circulation, and sympathetic nervous system output.
48. The method of any one of examples 42-47 wherein the patient's sympathetic nervous system output is monitored using an electrodermal sensor and/or one or more heart rate variability components.
49. The system of any one of examples 42-48 wherein the signal delivery element is configured to be positioned within the patients epidural space.
50. A system for treating acute decompensated heart failure (ADHF) in a patient, comprising:
an implantable electrical signal generator having a computer readable storage medium;
an implantable signal delivery element coupled to the signal generator, wherein the signal delivery element is configured to be positioned at least partially within the patient's epidural space at a target location within a vertebral range of T1 to T12, and wherein the signal delivery element is configured to apply about 1 kHz to about 100 kHz to neural tissue within the patient's epidural space; and
wherein the computer-readable storage medium has instructions that when executed:
determine a sympathetic activity level indicator that is the patient's splanchnic nerve activity; and
adjust the signal applied by the signal delivery element in response to the determined sympathetic activity level indicator.
51. The system of example 450, further comprising a sensor in communication with the computer-readable storage medium, wherein the sensor is configured to detect the activity of the one or more sympathetic nerves of the patient, and wherein the instructions, when executed, calculate the patient's sympathetic activity level.
52. The system of example 50 or example 51 wherein the instructions, when executed, and in response to a determined sympathetic activity level indicator greater than or equal to a predetermined target threshold, cease to apply the electrical signal.
53. The system of any one of examples 50-52 wherein the instructions, when executed, and in response to a determined sympathetic activity level indicator less than or equal to a predetermined target threshold, start application of the electrical signal.
54. The system of any one of examples 50-53 wherein the instructions, when executed, and in response to a determined sympathetic activity level indicator less than or equal to a predetermined target threshold, increase at least one of a frequency, an amplitude, or a pulse width of the electrical signal.
55. The method of any one of examples 50-54 wherein the patient's sympathetic activity is determined by monitoring one or more physiologic parameters selected from the group consisting of acute heart rate, chronic heart rate, lung congestion, edema, splanchnic circulation, and sympathetic nervous system output.
56. The method of any one of examples 50-55 wherein the patient's sympathetic nervous system output is monitored using an electrodermal sensor and/or one or more heart rate variability components.

1. A method for treating a patient having acute decompensated heart failure (ADHF) based at least on the patient having been diagnosed with ADHF, comprising:
positioning an implantable signal delivery device proximate to a target location at or near the patient's spinal cord within a vertebral range of about T1 to about T12; and
directing an electrical therapy signal to the target location via the implantable signal delivery device, wherein the electrical signal has a frequency in a frequency range of from 1.2 kHz to 100 kHz to modulate one or more of the patient's sympathetic nerves and treat the patient's ADHF.

2. The method of claim 1 wherein the electrical signal does not create paresthesia in the patient.

3. The method of claim 1 wherein the vertebral range is from about T5 to about T12.

4. The method of claim 1 wherein the electrical signal modulates the patient's splanchnic nerve activity.

5. The method of claim 4 wherein modulation of the patient's splanchnic nerve activity prevents and/or decreases venous congestion and/or pulmonary congestion.

6. The method of claim 4 wherein modulation of the patient's splanchnic nerve activity prevents and/or decreases fluid retention.

7. The method of claim 4 wherein modulation of the patient's splanchnic nerve activity increases the patient's splanchnic circulation.

8. The method of claim 4 wherein modulation of the patient's splanchnic nerve activity prevents and/or decreases lung congestion.

9. The method of claim 4 wherein modulation of the patient's splanchnic nerve activity prevents and/or decreases edema.

10. The method of claim 4 wherein modulation of the patient's splanchnic nerve activity reduces activity of the patient's sympathetic nervous system.

11. The method of claim 1 wherein the electrical signal further treats the patient's pain.

12. The method of claim 1 wherein the implantable signal delivery device is positioned in the patient's epidural space,

13. The method of claim 1, further comprising positioning a second implantable signal delivery device proximate to the target location.

14. The method of claim 13 wherein the implantable signal delivery device includes a paddle lead.

15. The method of claim 1 wherein the electrical therapy signal has a frequency of about 10 kHz.

16. The method of claim 1 wherein the electrical therapy signal has a pulse width of from about 20 microseconds to about 175 microseconds.

17. The method of claim 1 wherein the electrical therapy signal has an amplitude from about 20% of the patient's sensory threshold to about 90% of the patient's sensory threshold.

18. The method of claim 1 wherein the electrical therapy signal has an amplitude of from about 0.1 mA to about 20 mA.

19. The method of claim 1 wherein the one or more sympathetic nerves are sympathetic nerves associated with the patient's circulation.

20. The method of claim 1 wherein the one or more sympathetic nerves are selected from the group consisting of the greater splanchnic nerve, the lesser splanchnic nerve, and the least splanchnic nerve.

21. The method of claim 1 wherein modulating the one or more sympathetic nerves reduces mobilization of venous reservoirs, reduces splanchnic congestion, and/or reduces the patient's effective circulatory volume.

22. The method of claim 21 wherein splanchnic congestion is reduced by reducing the patient's retention of sodium and/or fluid.

23. The method of claim 1, further comprising:
monitoring the patient's ADHF by determining the patient's sympathetic activity; and
in response to results obtained from monitoring the patient's ADHF;
adjusting at least one signal delivery parameter in accordance with which the electrical signal is applied to the target location, wherein the signal delivery parameter is selected from the group consisting of frequency, amplitude, pulse width, duty cycle, and normal slow wave frequency, or
terminating delivery of the electrical therapy signal.

24. The method of claim 23 wherein the patient's sympathetic activity is determined by monitoring one or more physiologic parameters selected from the group consisting of acute heart rate, chronic heart rate, lung congestion, edema, splanchnic circulation, and sympathetic nervous system output.

25. The method of claim 23 wherein the patient's sympathetic nervous system output is monitored using an electrodermal sensor and/or one or more heart rate variability components.

26. A method for treating a patient having acute decompensated heart failure (ADHF) based at least on the patient having been diagnosed with ADHF, comprising:
monitoring at least one physiological parameter of the patient;
automatically detecting a change in the at least one physiological parameter that is outside of a predetermined threshold, wherein the change in the at least one physiological parameter is indicative of increased sympathetic nervous system activity in the patient; and
based on the detected parameter, delivering an electrical signal to the patient's spinal cord via at least one signal delivery element positioned in the patient's epidural space at a thoracic vertebral level from about T1 to about T12, the electrical signal having a frequency of from about 1.2 kHz to about 100 kHz.

27. The method of claim 26 wherein the thoracic vertebral level is from about T5 to about T12.

28. The method of claim 26 wherein the electrical signal does not create paresthesia in the patient.

29. The method of claim 26, further comprising delivering the electrical signal to the patient's spinal cord via the at least one signal delivery element positioned at the thoracic vertebral level.

30. The method of claim 26 wherein the electrical therapy signal has a frequency of about 10 kHz to 50 kHz.

31. The method of claim 26 wherein the electrical therapy signal has a pulse width of about 20 microseconds to about 175 microseconds,

32. The method of claim 26 wherein the electrical therapy signal has an amplitude from about 20% of the patient's sensory threshold to about 90% of the patient's sensory threshold,

33. The method of claim 26 wherein the electrical therapy signal has an amplitude of from about 0.1 mA to about 20 mA.

34. The method of claim 26 wherein the sympathetic nervous system comprises one or more sympathetic nerves.

35. The method of claim 34 wherein the one or more sympathetic nerves are sympathetic nerves associated with the patient's circulation.

36. The method of claim 34 wherein the one or more sympathetic nerves are selected from the group consisting of the greater splanchnic nerve, the lesser splanchnic nerve, and the least splanchnic nerve.

37. The method of claim 26 wherein the electrical signal modulates activity of one or more of the patient's splanchnic nerves.

38. The method of claim 26 wherein modulating the activity of one or more of the patient's splanchnic nerves reduces mobilization of venous reservoirs, reduces splanchnic congestion, and/or reduces the patient's effective circulatory volume.

39. The method of claim 38 wherein splanchnic congestion is reduced by reducing the patient's retention of sodium and/or fluid.

40. The method of claim 26 wherein the patient's sympathetic activity is determined by monitoring one or more physiologic parameters selected from the group consisting of acute heart rate, chronic heart rate, lung congestion, edema, splanchnic circulation, and sympathetic nervous system output.

41. The method of claim 40 wherein the patient's sympathetic nervous system output is monitored using an electrodermal sensor and/or one or more heart rate variability components.

42. The method of claim 26 wherein the electrical signal further treats the patient's pain.

43. The method of claim 26 wherein the electrical signal further treats the patient's ADHF.

44. A system for treating acute decompensated heart failure (ADHF) in a patient previously diagnosed with ADHF, comprising:
an implantable electrical signal generator having a computer readable storage medium;
an implantable signal delivery element coupled to the signal generator, wherein the signal delivery element is configured to be positioned at or proximate to the patient's spinal cord at a target location from about T1 to about T12, and wherein the signal delivery element is configured to apply about 1.2 kHz to about 100 kHz to the target location; and
wherein the computer-readable storage medium has instructions that when executed:
determine activity of one or more of the patient's sympathetic nerves; and
adjust the signal applied by the signal delivery element in response to the determined sympathetic nerve activity.

45. The system of claim 44, further comprising a sensor in communication with the computer-readable storage medium, wherein the sensor is configured to detect the activity of the one or more sympathetic nerves of the patient, and wherein the instructions, when executed, calculate the patient's sympathetic activity level.

46. The system of claim 45 wherein the instructions, when executed, and in response to a determined sympathetic activity level indicator greater than or equal to a predetermined target threshold, cease to apply the electrical signal.

47. The system of claim 45 wherein the instructions, when executed, and in response to a determined sympathetic activity level indicator less than or equal to a predetermined target threshold, start application of the electrical signal.

48. The system of claim 45 wherein the instructions, when executed, and in response to a determined sympathetic activity level indicator less than or equal to a predetermined target threshold, increase at least one of a frequency, an amplitude, or a pulse width of the electrical signal.

49. The system of claim 44 wherein the activity of one or more of the patient's sympathetic nerves is determined by monitoring one or more of the patient's physiologic parameters selected from the group consisting of acute heart rate, chronic heart rate, lung congestion, edema, splanchnic circulation, and sympathetic nervous system output.

50. The system of claim 49 wherein the patient's sympathetic nervous system output is monitored using an electrodermal sensor and/or one or more heart rate variability components.

51. The system of claim 44 wherein the signal delivery element is configured to be positioned within the patient's epidural space.

52. A system for treating acute decompensated heart failure (ADHF) in a patient previously diagnosed with ADHF, comprising:
an implantable electrical signal generator having a computer readable storage medium;
an implantable signal delivery element coupled to the signal generator, wherein the signal delivery element is configured to be positioned at least partially within the patient's epidural space at a target location within a vertebral range of T1 to T12, and wherein the signal delivery element is configured to apply about 1.2 kHz to about 100 kHz to neural tissue within the patient's epidural space;
a sensor in communication with the computer-readable storage medium, wherein the sensor is configured to detect the patient's sympathetic nerve activity, and wherein the instructions, when executed, calculate the patient's sympathetic activity level; and
wherein the computer-readable storage medium has instructions that when executed:
determine a sympathetic activity level indicator that is indicative of the patient's sympathetic activity level; and
adjust the signal applied by the signal delivery element in response to the determined sympathetic activity level indicator.

53. The system of claim 52 wherein the instructions, when executed, and in response to the determined sympathetic activity level indicator greater than or equal to a predetermined target threshold, cease to apply the electrical signal.

54. The system of claim 52 wherein the instructions, when executed, and in response to the determined sympathetic activity level indicator less than or equal to a predetermined target threshold, start application of the electrical signal.

55. The system of claim 52 wherein the instructions, when executed, and in response to the determined sympathetic activity level indicator less than or equal to a predetermined target threshold, increase at least one of a frequency, an amplitude, or a pulse width of the electrical signal.

56. The system of claim 52 wherein the patient's sympathetic activity is determined by monitoring one or more physiologic parameters selected from the group consisting of acute heart rate, chronic heart rate, lung congestion, edema, splanchnic circulation, and sympathetic nervous system output.

57. The system of claim 56 wherein the patient's sympathetic nervous system output is monitored using an electrodermal sensor and/or one or more heart rate variability components.
